(19) United States
(12) Patent Application Publication
(10) Pub. No.: US 2002/0072495 A1
(43) Pub. Date: Jun. 13, 2002

(54) LL-37 is an immunostimulant

(76) Inventors: Oleg Chertov, Frederick, MD (US); Joost J. Oppenheim, Bethesda, MD (US); De Yang, Frederick, MD (US); Glenn M. Anderson, Norristown, PA (US); Joseph M. Wooters, Brighton, MD (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/960,876

(22) Filed: Sep. 21, 2001

Related U.S. Application Data
(60) Provisional application No. 60/233,983, filed on Sep. 21, 2000.

(51) Int. Cl. A61K 38/17

(52) U.S. Cl. 514/12

(57) ABSTRACT

The present invention provides a method of enhancing an immune response in a subject, comprising administering an effective amount of LL-37. Moreover, the present invention provides a method of enhancing in a subject an immune response to a vaccine, comprising administering to the subject an effective amount of LL-37 with a vaccine. Further provided is a method of detecting a compound that decreases an immune response in a subject. A method of treating an autoimmune disease in a subject is thus provided. Also provided is a vaccine comprising an immunogen and LL-37.

This application claims priority to U.S. provisional application Ser. No. 60/233,983, filed Sep. 21, 2000, which is incorporated in its entirety herein.

### BACKGROUND OF THE INVENTION

1. Field of the Invention

The present invention relates to the field of immunology. In particular, the present invention relates to a method of enhancing an immune response in a subject and a method of treating an autoimmune disease in a subject.

2. Background Art

A variety of small (<100 amino acids) antimicrobial peptides, produced by cells of insects, plants, and animals, act as endogenous antibiotics. In humans, over a dozen of these antimicrobial peptides have thus far been identified and include several salivary histatins, lactoferricin, six α-defensins, two β-defensins, and an 18-kDa human cationic antimicrobial protein, hCAP18 (1). hCAP18, a protein possessing 140 amino acid residues (2-4), belongs to a family of proteins called cathelicidins which usually consist of a highly conserved preproregion of 128-143 residues including a putative 29-30 residue signal peptide and a 99-114 residue cathelin-like domain, and a C-terminal antimicrobial domain ranging in length from 12 to over 100 amino acid residues (5). The C-terminal domain of hCAP18 also has the capacity to bind and to neutralize bacterial LPS (2). Cleavage of hCAP18 was initially predicted (2, 3) and later confirmed (6) to occur between Ala103 and Leu104, giving rise to LL-37, a 37-residue mature antimicrobial peptide with two leucine residues on its N-terminus. LL-37/hCAP 18 is present in neutrophil granules (3) and is produced by bone marrow and testis (4), inflamed skin keratinocytes (7), lung epithelia (8), and squamous epithelia of human mouth, tongue, esophagus, cervix, and vagina (9). LL-37 is the only identified member in humans of a family of proteins called cathelicidins.

The present invention provides a human cathelicidin as an immunostimulant for the adaptive immune system.

### SUMMARY OF THE INVENTION

The present invention provides a method of enhancing an immune response in a subject, comprising administering to the subject an effective amount of LL-37, whereby the administration of LL-37 enhances an immune response in the subject.

The present invention further provides a method of enhancing in a subject an immune response to a vaccine, comprising administering to the subject an effective amount of LL-37 in combination with the vaccine, whereby the LL-37 enhances the immune response in the subject.

Further provided by the present invention is a method of detecting a compound that decreases an immune response in a subject, comprising a) contacting a monocyte or neutrophil or T cell migration system containing LL-37 with the compound and b) detecting a decrease in migration of monocytes or neutrophils or T cells in the system with the compound compared to migration of monocytes or neutrophils or T cells in the system without the compound, whereby the decrease in migration of monocytes or neutrophils or T cells in the system with the compound detects a compound that decreases an immune response in the subject.

The present invention provides a method of treating an autoimmune disease in a subject, comprising blocking LL-37 from binding to an FPRL1 receptor on a leukocyte, whereby blocking LL-37 from binding to the FPRL1 receptor decreases an autoimmune response in the subject, thereby treating the autoimmune disease.

Also provided by the present invention is a vaccine comprising LL-37.

### BRIEF DESCRIPTION OF THE DRAWINGS

FIGS. 1A-B show induction by LL-37 of migration of (A), and Ca²⁺ flux in (B), human monocytes.

A. The migration of monocytes (1×10⁶ cells/ml) was assessed by chemotaxis assay using 5-μm uncoated membranes. Spontaneous cell migration (without LL-37) was 30-50 cells/HPF. The average C.I. (Mean±SD) of triplicate wells is shown. *p<0.05 when compared with chemotaxis medium alone (open bar).

B. The arrow indicates the time point where LL-37 was applied to the cells.

FIGS. 2A-B show the effect of PTX (A) and serum (B) on LL-37-induced chemotaxis of monocytes.

A. Monocytes were incubated with (black bar) or without (hatched bar) PTX at a final concentration of 200 ng/ml for 30 min at 37° C. before performing chemotaxis assay. To show that the spontaneous cell migration (C.M.) was not affected by PTX pretreatment, the results are presented as No./HPF.

B. Chemotaxis assay was performed in the absence (hatched bar) or presence (black bar) of 10% human AB serum which can completely block the antimicrobial activity of LL-37 at 10⁻⁵ M.

FIGS. 3A-C show that LL-37 uses FPRL1 as its receptor.

A. Selective induction of FPRL1/293 cell chemotaxis by LL-37. The migration of parental HEK293 (open bars), FPRL1/293 (dotted bars), or ETFR (closed bars) cells was assessed by chemotaxis assay with the use of collagen-coated 10-μm membranes. Cells were used at a concentration of 1×10⁶ cells/ml. The average C.I. (Mean±SD) of triplicate wells is shown. Spontaneous cell migration (without LL-37) was 10-20 cells/HPF. *p<0.05 when compared with chemotaxis medium alone.

B. LL-37-induced Ca²⁺ flux in FPRL1/293 cells. The arrow indicates the time point where LL-37 was applied to the cells.

C. Cross-desensitization of LL-37-induced Ca²⁺ flux in monocytes by FPRL1-specific agonistic ligand. The arrows indicate the time points at which LL-37 and Su peptides were applied to the cells.

FIGS. 4A-B show the chemotaxis of human neutrophils (A) and CD4 T lymphocytes (B) in response to LL-37. The cell migration was assessed by chemotaxis assay with the use of uncoated (A) or fibronectin-coated (B) 5-μm membranes. The results are presented as the average C.I. (Mean±SD) of triplicate wells. *p<0.05 when compared with spontaneous cell migration (chemotactic medium alone). Neutrophils and CD4 T cells were used at a concentration of 1×10⁶ cells/ml and 5×10⁶ cells/ml, respectively. Spontaneous neutrophil and T cell migration (without LL-37) was 30-50 and 30-40 cells/HPF, respectively.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “a cell” includes multiple copies of the cell and can also include more than one particular species of cell.

The present invention provides a method of enhancing an immune response in a subject, comprising administering an effective amount of LL-37, whereby the administration of LL-37 enhances an immune response in the subject. As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human. In general, an “effective amount” of an agent is that amount needed to achieve the desired result or results, in this case, an enhanced immune response. An enhanced immune response is any detectable increase in a component of the subject's immune response. It can include, but is not limited to, an increase that improves the subject's clinical condition. A person of skill in the art can detect an enhanced immune response in a subject as taught in the Examples below.

By “immune response” is meant the response by the immune system of a subject to an organism, protein or other substance that is recognized as foreign to the subject. For example, a person of skill in the art knows that an immune response may include, but is not limited to, the mobilization of leukocytes to a site of entry into a subject's body by a foreign protein, virus or microbe; the release of antimicrobial substances by cells of the immune system; the release of chemotactic substances by cells of the immune system which attract other immune cells to the site of infection; and the release of chemokines by cells of the immune system, separately or in combination. An immune response can be humoral (i.e., the production of antibodies) and/or cellular.

By “innate immune response” is meant the pre-programmed host immune response, providing a first line defense against many microorganisms, based on recognition of common surface constituents of many different pathogens. By “adaptive immune response” is meant the acquired host immune response, characterized by replication and expansion of lymphocytes after encountering antigens specific for a particular microorganism. An “antigen” is a substance, usually a protein, capable of specifically binding to an antibody.

The method of the present invention can be used to enhance an immune response in a subject to bacterial infections, viral infections and cancer. The LL-37 binds to a formyl peptide receptor-like 1 receptor on monocytes to activate human neutrophils, monocytes and T cells.

The LL-37 can be administered to a subject via several routes, including but not limited to, oral administration, by inhalation, and by parenteral administration. Parenteral administration of LL-37, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, subcutaneous, intramuscular, intraperitoneal, intravenous and intratracheal routes. A more recently revised approach for parenteral administration involves use of a slow release or sustained release system such that a constant dosage is maintained. See, e.g., U.S. Pat. No. 3,610,795, which is incorporated by reference herein.

The LL-37 can be administered in a pharmaceutically acceptable carrier in doses in the range of about 0.1 mg to about 100 mg per day, depending on the sex and size of the subject and the underlying disease. Dosage schedules may be every 4 hours to once per day. By “pharmaceutically acceptable” is meant a material that is not biologically or otherwise undesirable, i.e., the material may be administered to an individual along with the LL-37 without causing substantial deleterious biological effects or interacting in a deleterious manner with any of the other components of the composition in which it is contained.

The present invention provides a method of enhancing in a subject an immune response to a vaccine, comprising administering to the subject an effective amount of LL-37 in combination with the vaccine, whereby the LL-37 enhances the immune response in the subject. By “in combination” is meant that the LL-37 can be administered before, contemporaneously with, or after the administration of the vaccine to the subject. Thus, LL-37 can be administered to the subject from 1-10 days before and/or after the injection of a vaccine to enhance the adaptive immune response of the subject. Moreover, LL-37 can be administered to a subject 1-10 days after the subject receives an injection comprising a vaccine and LL-37. Thus, LL-37 can act as an adjuvant. An “adjuvant” is a substance that non-specifically enhances the immune response to an immunogen. The LL-37 can be administered in combination with the vaccine, in a dose from about 0.1 mg to about 100 mg.

Thus, the present invention also provides a vaccine comprising an immunogen and LL-37. An “immunogen” is a substance, usually a protein, capable of producing an immune response and can be an antigen. In the vaccine of the invention, LL-37 can be combined with a tumor immunogen or an immunogen derived from an infectious agent, e.g., a bacterium, a virus or a parasite. Examples of tumor antigens include, but are not limited to, oncoproteins (products of oncogenes), e.g., p21^{ras}; differentiation antigens shared by melanomas and melanocytes, e.g., MART-1/MelanA, gp100, tyrosinase, TRP-1, TRP-2; and differentiation antigens shared on different tumors as well as normal testes, e.g., MAGE-1, MAGE-3, CAGE-1/2, BAGE, RAGE, NY-ESO-1 (30). Examples of viral immunogens that can be combined with LL-37 in a vaccine include, but are not limited to, immunogens derived from HIV; Hepatitis A, B, and C; polio; rubella; rubeola; and vaccinia. Bacterial immunogens derived from, for example, H. influenza, C. diphtheria and S. pneumoniae can also be combined with LL-37 in a vaccine.

The present invention also provides a method of detecting a compound that decreases an immune response in a subject, comprising a) contacting a leukocyte migration system containing LL-37 with the compound and b) detecting a decrease in migration of leukocytes in the system with the compound compared to migration of leukocytes in the system without the compound, whereby the decrease in migration of leukocytes in the system with the compound detects a compound that decreases an immune response in the subject. As is known to a person of skill in the art, a “leukocyte migration system” is a microchemotaxis chamber in which chemotactic factors diluted in chemotaxis medium are placed in wells of the lower compartment and leukocytes suspended in chemotaxis medium are added to the upper chamber (see Su et al., 1999, and Yang et al., 1999, incorporated by reference herein in their entirety). The upper and lower compartments are separated from each other by a polycarbonate membrane which traps migrating cells which can be stained and counted and presented as cells per high power field. A compound that decreases the migration of leukocytes toward the chamber containing a chemotactic factor decreases an immune response in a subject. As used herein, a “leukocyte” includes, but is not limited to, a monocyte, a neutrophil, a T cell, a basophil and an eosinophil.

Thus, the present invention provides a method of treating an autoimmune disease in a subject, by administering a compound that decreases an immune response in the subject. The method involves administering to the subject an effective amount of a compound detected according to the method above, whereby the administration of the compound treats an autoimmune disease in the subject. It is contemplated that a compound, identified according to the method above, competitively binds the formyl peptide receptor-like 1 (FPRL1) receptor on monocytes, thus blocking LL-37 from binding to the FPRL1 receptor, thereby decreasing the immune response in a subject.

Thus, the invention provides a method of treating autoimmune disease in a subject, comprising blocking LL-37 from binding to an FPRL1 receptor on a leukocyte, whereby blocking LL-37 from binding to the FPRL1 receptor decreases an autoimmune response in the subject, thereby treating the autoimmune disease.

Examples of autoimmune disease include, but are not limited to, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, CREST Syndrome, cold agglutinin disease, Crohn's Disease, discoid lupus, essential mixed cryoglobulinemia, fibromyalgia-fibromyositis, Graves' Disease, Guillain-Barré, Hashimoto's Thyroiditis, idiopathic pulmonary fibrosis, idiopathic thrombocytopenia purpura (ITP), IgA Nephropathy, insulin-dependent diabetes mellitus, juvenile arthritis, lichen planus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, Stiff-Man Syndrome, Takayasu Arteritis, temporal arteritis/giant cell arteritis, ulcerative colitis, uveitis, vasculitis, vitiligo and Wegener's Granulomatosis.

The following examples are put forth so as to provide those of ordinary skill in the art with a complete disclosure and description of how the compositions and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. The present invention is more particularly described in the following examples which are intended as illustrative only since numerous modifications and variations therein will be apparent to those skilled in the art.

### EXAMPLES

Dulbecco's Modified Eagle's Medium (DMEM) and RPMI 1640 were purchased from Biowhittaker (Walkersville, Md.). Synthetic fMLP and human AB serum were purchased from Sigma (St. Louis, Mo.). FBS was purchased from Hyclone (Logan, Utah). Su peptide, a shorter version (lacking the N-terminal 5 amino acids) of T21/DP107 corresponding to aa 563-595 of HIV envelope protein gp41 (17), was synthesized and purified by Macromolecular Resources (Fort Collins, Colo.). Su peptide is a specific agonist for formyl peptide receptor-like 1 (FPRL1) receptor. Su peptide was 99% pure as verified by mass spectrometry. LL-37 was synthesized by solid-phase Fmoc (9-fluorenylmethyloxycarbonyl)-chemistry. The first 11 amino acids were each coupled once and the remaining amino acids were doubly coupled. After cleavage from the resin, the peptide was dissolved in Buffer A and purified by reverse-phase chromatography on a Dynamax C18 HPLC column using a gradient of 5-30% B in A buffer in 10 min, followed by 30-100% B in 90 min at a flow rate of 45 ml/min (Buffer A: 0.1% TFA in water; Buffer B: 0.1% TFA in acetonitrile). The fractions were characterized by analytical HPLC, mass spectrometry and capillary electrophoresis, combined and lyophilized. The LL-37 peptide thus prepared was 99.9% pure. Both LL-37 and Su peptide solutions were free of endotoxin.

Cells:

Human peripheral blood mononuclear cells (PBMC) were isolated from leukopacks (Courtesy of the Transfusion Medicine Department, NIH Clinic Center, Bethesda, Md.) by routine Ficoll-Hypaque density gradient centrifugation. Monocytes were purified from human PBMC with the use of MACS CD 14 monocyte isolation kit (Miltenyi Biotech Inc., Auburn, Calif.) according to the manufacturer's recommendation (12). Neutrophils were purified from the same leukopacks by 3% dextran sedimentation. T cells were purified from PBMC by the use of Human CD4 T Cell Enrichment Columns (R & D Systems, Minneapolis, Minn.) (12). The purity of neutrophils, monocytes, and T cells by FACScan analysis was more than 95%. Rat basophilic leukemia cells stably transfected with epitope-tagged formyl peptide receptor (ETFR cells) were generously provided by Drs. H. Ali and R. Snyderman (Duke University, Durham, N.C.), and maintained in the presence of 0.8 mg/ml of geneticin in DMEM supplemented with 10% FBS. Human embryonic kidney cells 293 stably transfected with FPRL1 (designated FPRL1/293 cells thereafter) were a gift of Drs. P. M. Murphy and J. L. Gao (NIAID, NIH, Bethesda, Md.) and were maintained in the presence of 2 mg/ml of geneticin in DMEM supplemented with 10% FBS.

Chemotaxis assay:

Migration of neutrophils, monocytes, T cells, and HEK293 or transfectant cells in response to chemotactic factors was assessed using a 48-well microchemotaxis chamber technique as previously described (17, 18). Chemotactic factors diluted in chemotaxis medium (CM, RPMI 1640 containing 1% BSA) were placed in the wells of the lower compartment of the chamber (Neuro Probe, Cabin John, Mass.), and cells suspended in CM were added to the upper compartment. The lower and upper compartments were separated by a polycarbonate membrane (Osmonics, Livermore, Calif.). After incubation at 37° C. in humidified air with 5% CO2 for a period of time (60 min for neutrophils, 90 min for monocytes, 180 min for T cells, and 300 min for HEK293, ETFR, and FPRL1/293 cells), the membranes were removed, scraped, stained, and counted. The results are presented as number of cells per high power field (No./HPF) or as chemotactic index (C.I.).

Ca²⁺ mobilization assay:

Monocytes or FPRL1/293 cells (10⁷ cells/ml in RPMI 1640 containing 10% FBS) were loaded by incubating with 5 μM Fura-2 (Molecular Probe) at 24° C. for 30 minutes in the dark. Subsequently, the loaded cells were washed with and resuspended (10⁶ cells/ml) in saline buffer (138 mM NaCl, 6 mM KCl, 1 mM CaCl₂, 10 mM HEPES, 5 mM glucose, and 1% BSA, pH 7.4). Each 2 ml of loaded cells suspension was then transferred into a quartz cuvette, which was placed in a luminescence spectrometer LS50 B (Perkin-Elmer Limited, Beaconsfield, U.K). Ca²⁺ mobilization of the cells in response to chemotactic factors was measured by recording the ratio of fluorescence emitted at 510 nm after sequential excitation at 340 nm and 380 nm.

Statistical analysis:

Unless otherwise specified, all experiments were performed at least three times and the results show representative experiments. The significance of the difference between test and control groups was analyzed with a Student's t test.

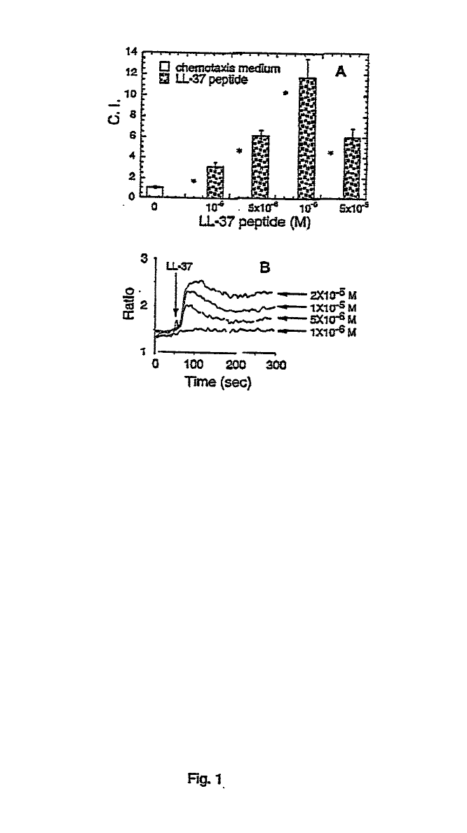

In vitro migration of human peripheral blood monocytes is a critical correlate of monocyte accumulation at sites of inflammation or injury. LL-37 induced monocyte migration in a dose-dependent manner with an optimal concentration at 10⁻⁵ M (FIG. 1A). In addition, LL-37 also dose-dependently induced Ca²⁺ flux in monocytes (FIG. 1B). A checkerboard analysis revealed that when equal concentrations of LL-37 were present in both the lower and the upper wells, only a slight increase in cell migration was observed (Table I). Thus, the migration of monocytes induced by LL-37 was based predominantly on chemotaxis rather than chemokinesis. Interestingly, after monocytes were differentiated into immature dendritic cells, the immature dendritic cells could not respond to LL-37 in terms of either chemotaxis or Ca²⁺ mobilization presumably due to the loss of functional expression of the receptor for LL-37.

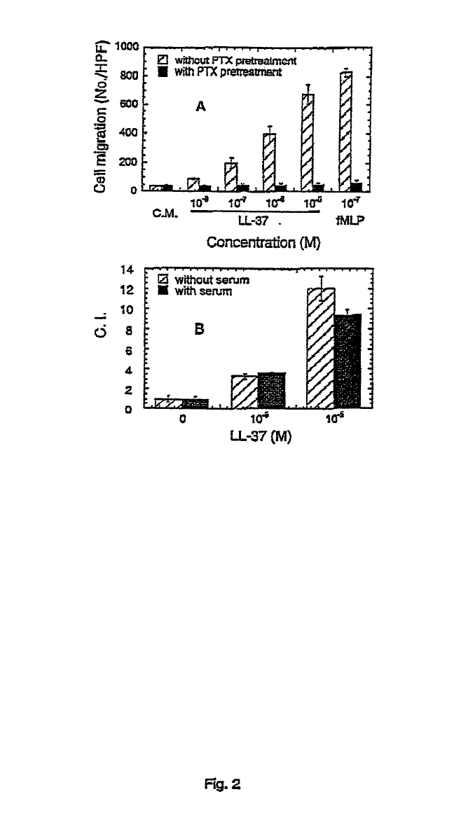

To characterize the nature of the receptor for LL-37, the effect of pertussis toxin (PTX), a reagent known to selectively block Gi protein-coupled signaling, on LL-37-induced monocyte chemotaxis was examined. Treatment of monocytes with PTX (200 ng/ml) at 37° C. for 30 min nearly completely inhibited subsequent cell migration in response to LL-37, indicating that a Gi-protein-coupled receptor might be involved in LL-37-induced monocyte activation (FIG. 2A).

The only previously known effect of LL-37 on mammalian cells is its capacity to damage them at concentrations above 5×10⁻⁵ M. This cytocidal effect of LL-37 can be blocked completely by the presence of human serum and partially by porcine serum (10). To investigate the relationship of the chemotactic effect of LL-37 to its cytocidal effect, the influence of 10% human AB serum on LL-37-induced chemotaxis was studied. LL-37-induced chemotaxis of monocytes was not significantly inhibited by the presence of 10% human AB serum (FIG. 2B), suggesting that the chemotactic effect of LL-37 was independent of its cytocidal effect.

To mediate its chemotactic effect on monocytes, LL-37 must use a chemotactic receptor expressed by monocytes, but not by monocyte-derived immature dendritic cells. Of the 22 human chemotactic receptors (including 17 chemokine receptors and 5 classical chemoattractant receptors) identified so far (19, 20), nine, namely FPR, FPRL1, PAFR, C5aR, CXCR4, CCR1, CCR2, CCR5, and CCR8, have been demonstrated to be expressed by freshly isolated peripheral blood monocytes at mRNA, protein, and functional levels (19-21). Seven out of the nine (FPR, PAFR, C5aR, CXCR4, CCR1, CCR5, and CCR8) have also been shown to be functionally expressed by monocyte-derived immature dendritic cells (21, 22). Examination of HEK293 cells stably transfected to express CCR2 indicated that CCR2 was not a receptor for LL-37.

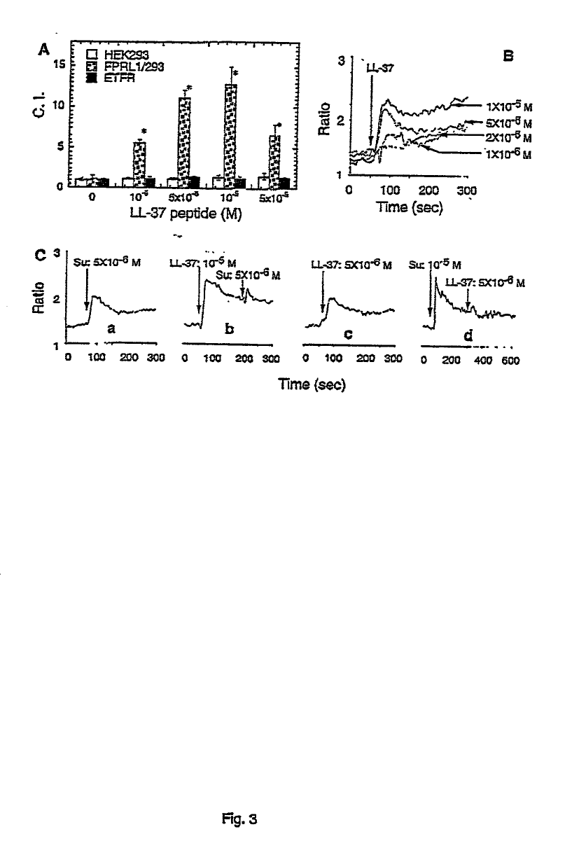

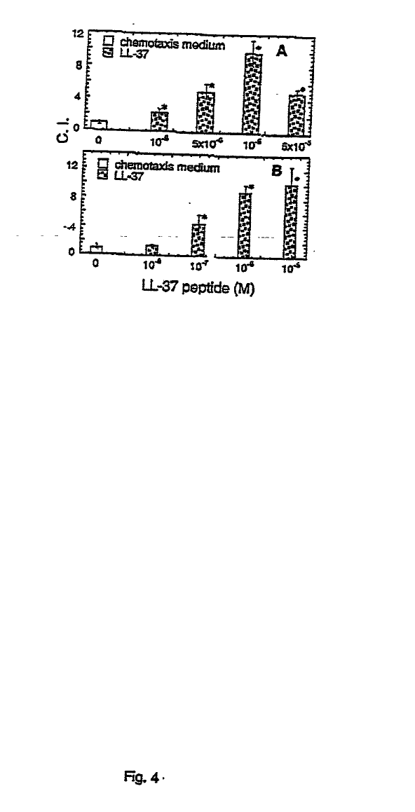

FPRL1 was studied to determine whether it serves as a receptor for LL-37. As shown by FIG. 3A, FPRL1/293 cells, HEK293 cells transfected to stably express FPRL1, migrated in response to LL-37 in a dose-dependent manner, whereas the parental HEK293 cells did not respond, suggesting FPRL1 is a receptor for LL-37. The specificity of LL-37 interaction with FPRL1 is further supported by the fact that cells expressing formyl peptide receptor (ETFR cells), which shares the highest homology with FPRL1, also did not respond to LL-37 (FIG. 3A). LL-37 also induced Ca²⁺ flux in a dose-dependent manner in FPRL1/293 cells (FIG. 3B), but not in ETFR cells.

In order to ascertain that LL-37 also utilizes FPRL1 as a receptor to mediate its effect on monocytes, LL-37-induced Ca²⁺ flux in monocytes was studied to determine whether it could be cross-desensitized by Su peptide, an FPRL1-specific agonist. As shown by FIG. 3C, both Su peptide and LL-37 at 5×10⁻⁶ M induced Ca²⁺ flux to a similar extent in freshly isolated human peripheral blood monocytes (a and c, respectively). Furthermore, the Ca²⁺ flux induced by Su peptide at 5×10⁶ M was almost completely desensitized by LL-37 at 10⁻⁵ M (FIGS. 3C, b), and vice versa (FIGS. 3C, d). Since cross-desensitization of Ca²⁺ flux is often due to two agonists acting on the same receptor, it appears that LL-37 utilizes FPRL1 as a functional receptor. LL-37 was also examined to determine whether it could chemoattract freshly isolated human peripheral blood neutrophils and T lymphocytes that are known to express functional FPRL1 (17, 19). LL-37 induced dose-dependent migration of highly purified human neutrophils (FIG. 4A) and CD4 T cells (FIG. 4B).

LL-37 is chemotactic for freshly isolated human peripheral blood neutrophils, monocytes, and T cells and utilizes FPRL1 as a receptor to mediate its chemotactic and Ca²⁺-mobilizing effects. Because leukocytes participate in both innate and adaptive immunity, the fact that LL-37 can chemoattract human leukocytes provides one additional new mechanism by which LL-37 contributes to host defense against microbial invasion, by participating in the recruitment of leukocytes to sites of infection. This mechanism is potentially important in vivo since the chemotactic activity of LL-37, unlike its antimicrobial action (10), is not significantly inhibited by the presence of 10% human AB serum (FIG. 2B).

The optimal concentration for LL-37 to chemoattract human leukocytes was 10⁻⁵ M (FIGS. 1A & 4). Most chemotactic responses are usually based on “high-affinity ligand-receptor interaction” (19, 20). It is likely that LL-37-FPRL1 interaction represents a low-affinity ligand-receptor interaction. Previously identified low-affinity ligand-receptor interactions include multiple CXC chemokines (GRO, NAP2, and ENA-78) for CXCR1 (23), and MIP-1β and MCP-1 for CCR1 (24). Low-affinity ligand-receptor interactions also contribute to the recruitment of leukocytes to the focus of inflammation (25). Multiple leukocyte-chemotactic factors including LL-37 are likely to be generated at sites of microbial invasion and presumably form a complex of concentration gradients. As leukocytes traffic from the lower to the higher levels of the chemotactic gradients, their high-affinity chemotactic receptors are likely to become deactivated through desensitization (25). Subsequent low-affinity ligand-receptor interactions, such as LL-37-FPRL1 interaction, can thus direct leukocytes closer to inflammatory foci. The concentration of LL-37 in the fluid from xenografts prepared with normal human airway epithelia is 2 μg/ml, equivalent to 4×10⁻⁷ M (26). Assuming that airway inflammation could also induce a 50-fold increase in LL-37 expression, the LL-37 at the site of airway inflammation would reach 2×10⁻⁵ M. Thus, LL-37 can potentially reach its optimal chemotactic concentration (10⁻⁵ M) at local inflammatory sites.

In addition to LL-37, other known ligands for FPRL1 include bacterial formyl peptides (19), an ectodomain of HIV gp41 termed T21 (17), a hexapeptide (Trp-Lys-Tyr-Met-Val-D-Met-NH2) termed W peptide (27), serum amyloid A (28), and even lipoxin A4, a lipid derivative of arachidonate metabolism (29). Of these, serum amyloid A, lipoxin A4, and LL-37 are endogenously generated and increase dramatically during inflammation (7, 9, 28, 29). Activation of the chemotactic receptor FPRL1 by these endogenous ligands including LL-37 results in a G-protein-mediated signaling cascade leading not only to chemotaxis of leukocytes, but also increased adhesion, enhanced phagocytosis, release of oxygen intermediates. and augmented bacterial killing (19, 20), thereby promoting antimicrobial immunity. Because LL-37 can also bind and neutralize bacterial LPS (2), it may, on the other hand, limit the adverse effects of inflammation by down-regulating LPS-induced inflammatory responses.

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

### References

1. Lehrer, R. I., and T. Ganz. 1999. Antimicrobial peptides in mammalian and insect host defense. Curr. Opin. Immunol. 11:23-27.

2. Larrick, J. W., M. Hirata, R. F. Balint, J. Lee, J. Zhong, and S. C. Wright. 1995. Human CAP18: a novel antimicrobial lipopolysaccharide-binding protein. Infect. Immun. 63:1291-1297.

3. Cowland, J. B., A. H. Johnsen, and N. Borregaad. 1995. hCAP-18, a cathelin/pro-bactenecin-like protein of human neutrophil specific granules. FEBS Lett. 368:173-176.

4. Agerberth, B., H. Gunne, J. Odeberg, P. kogner, H. G. Boman, and G. H. Gudmundsson. 1995. FALL-39, a putative human peptide antibiotic, is cysteine-free and expressed in bone marrow and testis. Proc. Natl. Acad. Sci. USA 92:195-199.

5. Zanetti, M., R. Gennaro, and D. Romeo. 1995. Cathelicidins: a novel protein family with a common proregion and a variable C-terminal antimicrobial domain. FEBS Lett. 374:1-5.

6. Gudmundsson, G. H., B. Agerberth, J. Odeberg, T. Bergman, B. Olsson, and R. Salcedo. 1996. The human gene FALL-39 and processing of the cathelin precursor to the antibacterial peptide LL-37 in granulocytes. Eur. J. Biochem. 238:325-332.

7. Frohm, M., B. Agerberth, G. Ahangari, M. Stahle-Backdahl, S. Liden, H. Wigzell, and G. H. Gudmundsson. 1997. The expression of the gene coding for the antimicrobial peptide LL-37 is induced in human keratinocytes during inflammatory disorders. J. Biol. Chem. 272:15258-15263.

8. Bals, R., X. Wang, M. Zasloff, and J. M. Wilson. 1998. The peptide antibiotic LL-37/hCAP-18 is expressed in epithelia of the human lung where it has broad antimicrobial activity at the airway surface. Proc. Natl. Acad. Sci. USA 95:9541-9546.

9. Nilsson, M. F., B. Sandstedt, 0. Sorensen, G. Weber, N. Borregaad, and M. Stahle-Backdahl. 1999. The human cationic antimicrobial protein (hCAP18), a peptide antibiotic, is widely expressed in human squamous epithelia and colocalizes with interleukin-6. Infect. Immun. 67:2561-2566.

10. Johansson, J., G. H. Gudmundsson, M. E. Rottenberg, K. D. Bemdt, and B. Agerberth. 1998. Conformation-dependent antibacterial activity of naturally occurring human peptide LL-37. J. Biol. Chem. 273:3718-3724.

11. Chertov, O., D. F. Michiel, L. Xu, J. M. Wang, K. Tani, W. J. Murphy, D. L. Longo, D. D. Taub, and J. J. Oppenheim. 1996. Identification of defensin-1, defensin-2, and CAP37/azurocidin as T-cell chemoattractant proteins released from interleukin-8-stimulated neutrophils. J. Biol. Chem. 271:2935-2940.

12. Yang, D., Q. Chen, 0. Chertov, and J. J. Oppenheim. 2000. Human neutrophil defensins selectively chemoattract naïve T and immature dendritic cells. J. Leukoc. Biol. 68:9-14.

13. Lillard, Jr., J. W., P. N. Boyaka, 0. Chertov, J. J. Oppenheim, and J. R. McGhee. 1999. Mechanisms for induction of acquired host immunity by neutrophil peptide defensins. Proc. Natl. Acad. Sci. USA 96:651-656.

14. Yang, D., O. Chertov, S. N. Bykovskaia, Q. Chen, M. J. Buffo, J. Shogan, M. Anderson, J. M. Schroder, J. M. Wang, O. M. Z. Howard, and J. J. Oppenheim. 1999. β-Defensins: Linking innate and adaptive immunity through dendritic and T cell CCR6. Science 286:525-528.

15. Chertov, O., H. Ueda, L. L. Xu, K. Tani, W. J. Murphy, J. M. Wang, O. M. Z. Howard, T. J. Sayers, and J. J. Oppenheim. 1997. Identification of human neutrophil-derived cathepsin G and azurocidin/CAP37 as chemoattractants for mononuclear cells and neutrophils. J. Exp. Med. 186:739-747.

16. Huang, H. J., C. R. Ross, and F. Blecha. 1997. Chemoattractant properties of PR-39, a neutrophil antibacterial peptide. J. Leukoc. Biol. 61:624-629.

17. Su, S. B., J. -L. Gao, W. -H. Gong, N. M. Dunlop, P. M. Murphy, J. J. Oppenheim, and J. M. Wang. 1999. T21/DP107, a synthetic leucine zipper-like domain of the HIV-1 envelope gp41, attracts and activates human phagocytes by using G-protein-coupled formyl peptide receptors. J. Immunol. 162:5924-5930.

18. Yang, D., O. M. Z. Howard, Q. Chen, and J. J. Oppenheim. 1999. Cutting Edge: Immature dendritic cells generated from monocytes in the presence of TGF-β1 express functional C-C chemokine receptor 6. J. Immunol. 163:1737-1741.

19. Murphy, P. M. 1994. The molecular biology of leukocyte chemoattractant receptors. Annu. Rev. Immunol. 12:593-633.

20. Zlotnick, A., J. Morales, and J. A. Hedrick. 1999. Recent advances in chemokines and chemokine receptors. Crit. Rev. Immunol. 19:1-47.

21. Sozzani, S., P. Allavena, A. Vecchi, and A. Mantovani. 1999. The role of chemokines in the regulation of dendritic cell trafficking. J. Leukoc. Biol. 66:1-9.

22. Yang, D., Q. Chen, Y. Le, J. M. Wang and J. J. Oppenheim. 2001. Differential regulation of formyl peptide receptor-like 1 expression during the differentiation of monocytes to dendritic cells and macrophages. J. Immunol. 166:4092-8.

23. Ahuja, S. K., and P. M. Murphy. 1996. The CXC chemokines growth-regulated oncogene (GRO) alpha, GRO beta, GRO gamma, neutrophil-activating peptide-2, and epithelial cell-derived neutrophil-activating peptide-78 are potent agonists for the type B, but not the type A, human interleukin-8 receptor. J. Biol. Chem. 271:20545-20550.

24. Neote, K., D. DiGregorio, J. Y. Mak, R. Horuk, and T. J. Schall. 1993. Molecular cloning, functional expression, and signaling characteristics of a C-C chemokine receptor. Cell 72:415-425.

25. Foxman, E. F., J. J. Campbell, and E. C. Butcher. 1997. Multi-step navigation and the combinatorial control of leukocyte chemotaxis. J. Cell Biol. 139:1349-1360.

26. Bals, R., D. J. Weiner, R. L. Meegalla, and J. M. Wilson. 1999. Transfer of a cathelicidin peptide antibiotic gene restores bacterial killing in a cystic fibrosis xenograft model. J. Clin. Invest. 103:1113-1117.

27. Le, Y., W. -H. Gong, B. Li, N. M. Dunlop, W. Shen, S. B. Su, R. D. Ye, and J. M. Wang. 1999. Utilization of two seven-transmembrane, G protein-coupled receptors, formyl peptide receptor-like 1 and formyl peptide receptor, by the synthetic hexapeptide WKYMVm for human phagocyte activation. J. Immunol. 163:6777-6784.

28. Su, S. B., W. -h. Gong, J. -L. Gao, W. Shen, P. M. Murphy, J. J. Oppenheim, and J. M. Wang. 1999. A seven-transmembrane, G protein-coupled receptor, FPRL1, mediates the chemotactic activity of serum amyloid A for human phagocytic cells. J. Exp. Med. 189:395-402.

29. Fiore, S., J. F. Maddox, H. D. Peres, and C. N. Serhan. 1994. Identification of a human cDNA encoding a functional high affinity lipoxin A4 receptor. J. Exp. Med. 180:253-260.

30. Rosenberg, S. A. A new era for cancer immunotherapy based on the genes that encode cancer antigens. Immunity 10(3):281-7.

TABLE I
Checkerboard analysis of LL-37 induced monocyte migration

| LL-37 in the | | | |
|---|---|---|---|
| lower wells | LL-37 in the upper wells (μM) | | |
| (μM) | 0 | 1 | 10 |
| 0 | 40 ± 5 | 45 ± 6 | 44 ± 7 |
| 1 | 135 ± 15** | 50 ± 8 | 40 ± 6 |
| 10 | 495 ± 29** | 265 ± 5** | 95 ± 2* |

Human monocytes were used at 1×10⁶/ml. LL-37 peptide at the specified concentrations was added to the lower wells of the chemotaxis chamber, and monocytes in the absence or presence of the specified concentration of LL-37 peptide were added to the upper wells of the chemotaxis chamber. The average (Mean±SD) of migrated monocytes of triplicated wells is shown as No./HPF. *p<0.05 and **p<0.01 as compared with spontaneous monocyte migration (in bold type).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of enhancing an immune response in a subject, comprising administering to the subject an effective amount of LL-37, whereby the administration of LL-37 enhances an immune response in the subject.

2. The method of claim 1, wherein the immune response is an adaptive immune response.

3. The method of claim 1, wherein the subject is a mammal.

4. The method of claim 3, wherein the mammal is a human.

5. A method of enhancing in a subject an immune response to a vaccine, comprising administering to the subject an effective amount of LL-37 in combination with the vaccine, whereby the LL-37 enhances the immune response in the subject.

6. The method of claim 5, wherein the subject is a mammal.

7. The method of claim 6, wherein the mammal is a human.

8. A method of detecting a compound that decreases an immune response in a subject, comprising:
a) contacting a leukocyte migration system containing LL-37 with the compound; and
b) detecting a decrease in migration of leukocytes in the system with the compound compared to migration of leukocytes in the system without the compound, whereby the decrease in migration of leukocytes in the system with the compound detects a compound that decreases an immune response in the subject.

9. The method of claim 8, wherein the leukocytes are selected from the group consisting of monocytes, neutrophils and T cells.

10. The method of claim 8, wherein the subject is a mammal.

11. The method of claim 10, wherein the mammal is a human.

12. A method of treating an autoimmune disease in a subject, comprising administering to the subject an effective amount of a compound detected according to the method of claim 8, whereby the administration of the compound treats an autoimmune disease in the subject.

13. The method of claim 12, wherein the autoimmune disease comprises autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, CREST Syndrome, cold agglutinin disease, Crohn's Disease, discoid lupus, essential mixed cryoglobulinemia, fibromyalgia-fibromyositis, Graves' Disease, Guillain-Barré, Hashimoto's Thyroiditis, idiopathic pulmonary fibrosis, idiopathic thrombocytopenia purpura (ITP), IgA Nephropathy, insulin-dependent diabetes mellitus, juvenile arthritis, lichen planus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, Stiff-Man Syndrome, Takayasu Arteritis, giant cell arteritis, ulcerative colitis, uveitis, vasculitis, vitiligo and Wegener's Granulomatosis.

14. The method of claim 12, wherein the subject is a mammal.

15. The method of claim 14, wherein the mammal is a human.

16. A method of treating an autoimmune disease in a subject, comprising blocking LL-37 from binding to an FPRL1 receptor on a leukocyte, whereby blocking LL-37 from binding to the FPRL1 receptor decreases an autoimmune response in the subject, thereby treating the autoimmune disease.

17. The method of claim 16, wherein the autoimmune disease comprises autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, CREST Syndrome, cold agglutinin disease, Crohn's Disease, discoid lupus, essential mixed cryoglobulinemia, fibromyalgia-fibromyositis, Graves' Disease, Guillain-Barré, Hashimoto's Thyroiditis, idiopathic pulmonary fibrosis, idiopathic thrombocytopenia purpura (ITP), IgA Nephropathy, insulin-dependent diabetes mellitus, juvenile arthritis, lichen planus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis, dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, Stiff-Man Syndrome, Takayasu Arteritis, giant cell arteritis, ulcerative colitis, uveitis, vasculitis, vitiligo and Wegener's Granulomatosis.

18. The method of claim 16, wherein the subject is a mammal.

19. The method of claim 18, wherein the mammal is a human.

20. A vaccine comprising an immunogen and LL-37.

21. The vaccine of claim 20, wherein the immunogen is selected from the group consisting of p21^{ras}, MART-1/MelanA, gp100, tyrosinase, TRP-1, TRP-2, MAGE-1, MAGE-3, CAGE-1/2, BAGE, RAGE, NY-ESO-1, HIV, Hepatitis A, Hepatitis B, Hepatitis C, polio, rubella, rubeola, vaccinia, H. influenza, C. diphtheria and S. pneumoniae.
